(19) United States
(12) Patent Application Publication
(10) Pub. No.: US 2026/0130683 A1
(43) Pub. Date: May 14, 2026

(54) TREATMENT TOOL FOR ENDOSCOPE AND ENDOSCOPE SYSTEM

(71) Applicant: FUJIFILM Corporation, Tokyo (JP)

(72) Inventor: Yoshihiro UEDA, Ashigarakami-gun (JP)

(73) Assignee: FUJIFILM Corporation, Tokyo (JP)

(21) Appl. No.: 19/382,263

(22) Filed: Nov. 7, 2025

Related U.S. Application Data
(60) Provisional application No. 63/720,697, filed on Nov. 14, 2024.

(51) Int. Cl. A61B 17/29; A61B 1/018; A61B 17/00

(52) U.S. Cl. CPC A61B 17/2909; A61B 1/018; A61B 2017/00477; A61B 2017/2903; A61B 2017/2905; A61B 2017/2908; A61B 2017/2929; A61B 2017/294

(57) ABSTRACT

A treatment tool for an endoscope, includes: an insertion part that is insertable into a body and that includes a distal end part having an openable and closable treatment part; an operation part that is disposed on a base end side of the insertion part; and at least one operation wire that is connected to the operation part and that operates opening and closing of the treatment part, the treatment part includes a distal end operation part, a link member that is rotationally connected to a base end side of the distal end operation part, and a connecting member that is rotationally connected to a base end side of the link member, the operation wire is fixed to the connecting member, and at least a part of the operation wire and the link member overlap each other in a longitudinal axis direction of the insertion part.

### CROSS REFERENCE TO RELATED APPLICATION

This application is based on and claims priority from US Provisional Application No. 63/720,697 filed on November 14, 2024, the entire disclosure of which is incorporated herein by reference.

### BACKGROUND OF THE INVENTION

### 1. Field of the Invention

The present invention relates to a treatment tool for an endoscope and an endoscope system.

### 2. Description of the Related Art

JP1993-285147A (JP-H05-285147A) discloses grip forceps including a metal sheath to be inserted into a body cavity, a grip part provided at a distal end of the sheath, an operation part provided at a base end part of the sheath and operated for opening and closing the grip part, a fixing screw for fixing the operation part to the sheath, and an electrode pin provided integrally with the fixing screw to cause a high-frequency current to flow to the grip part through the sheath.

JP1998-118089A (JP-H10-118089A) discloses a treatment tool for an endoscope in which a rear end part of an insertion member to be inserted into a body via an endoscope is connected to an operation mechanism, an operation member has a front end part on which a closing/opening body is supported to be movable rotationally, a interlocking member is provided in the insertion member, a rear end part of the interlocking member is drivingly connected to the operation mechanism, a front end part of the interlocking member is drivingly connected to the closing/opening body via a linear member, the closing/opening body is operated based on remote operation of the operation mechanism, the linear member is made single, and the closing/opening body is biased in a direction in which the closing/opening body is opened by an elastic force of the linear member.

JP2013-042921A discloses a cardiac stimulation device system comprising an electrode that has a substrate formed in a sheet shape from an insulating material and having a through-part formed to penetrate therethrough in a thickness direction and a conductive application part attached to one surface of the substrate, and an electrode operation treatment tool having a distal end part to which the electrode is attachably and detachably attached, in which the electrode operation treatment tool has a retraction unit that retracts a pericardium on one surface side of the substrate into the through-part in a state where the electrode is attached to the distal end part, and the electrode has a locking member that protrudes into the through-part along the one surface.

JP2000-070280A discloses a high-frequency treatment tool comprising an insertion part that is insertable into a body, a pair of grip parts that are disposed at a distal end part of the insertion part and that grip a biological tissue to perform coagulation or incision, and an operation part that is connected to a base end part of the insertion part and that is operated for opening and closing the grip parts, in which the biological tissue gripped by the grip parts is coagulated or incised by applying a high-frequency current to electrode parts provided on grip surfaces of the grip parts, an electrical insulation part is provided on the grip surface of at least one of the grip parts, a predetermined gap is formed between the grip surfaces of the grip parts by bringing the electrical insulation part into contact with the grip surface of the other grip part in a state where the grip parts are completely closed, and a short circuit between the electrode parts of the grip parts is prevented.

### SUMMARY OF THE INVENTION

One embodiment according to the technology of the present disclosure provides a treatment tool for an endoscope and an endoscope system capable of improving operability by shortening a rigid portion at a distal end.

(1)

A treatment tool for an endoscope, comprising:

an insertion part that is insertable into a body and that includes a distal end part having an openable and closable treatment part;

an operation part that is disposed on a base end side of the insertion part; and

at least one operation wire that is connected to the operation part and that operates opening and closing of the treatment part,

in which the treatment part includes a distal end operation part, a link member that is rotationally connected to a base end side of the distal end operation part, and a connecting member that is rotationally connected to a base end side of the link member,

the operation wire is fixed to the connecting member, and

at least a part of the operation wire and the link member overlap each other in a longitudinal axis direction of the insertion part.

(2)

The treatment tool for an endoscope according to (1),

in which the connecting member has a rotation shaft portion that rotationally connects the link member and the connecting member.

(3)

The treatment tool for an endoscope according to (2),

in which the connecting member has the rotation shaft portion and an insertion hole through which the operation wire is insertable and which intersects with a rotation axis of the rotation shaft portion, and

the operation wire is fixed to an inner surface of the insertion hole.

(4)

The treatment tool for an endoscope according to (3),

in which the insertion hole is longer than the rotation shaft portion in the longitudinal axis direction.

(5)

The treatment tool for an endoscope according to (4),

in which a portion where the inner surface of the insertion hole and the operation wire are in contact with each other is longer than the rotation shaft portion in the longitudinal axis direction.

(6)

The treatment tool for an endoscope according to any one of (2) to (5), further comprising:

a housing that houses the connecting member and that has a guide groove for guiding a movement of the rotation shaft portion in the longitudinal axis direction.

(7)

The treatment tool for an endoscope according to any one of (1) to (6),

in which the insertion part includes a bendable part that is bendable and that is provided to be adjacent to the distal end part on a side of the operation part.

(8)

The treatment tool for an endoscope according to (7),

in which the bendable part is bent by an operation of the operation part.

(9)

The treatment tool for an endoscope according to (1),

in which the connecting member and the operation wire are fixed by laser welding.

(10)

The treatment tool for an endoscope according to (9),

in which the connecting member and the operation wire are laser-welded at a location other than on a rear end surface of the connecting member.

(11)

An endoscope system comprising:

the treatment tool for an endoscope according to any one of (1) to (10); and

an endoscope having a treatment tool channel through which the insertion part is insertable.

According to the present invention, it is possible to provide a treatment tool for an endoscope and an endoscope system capable of improving operability by shortening a rigid portion at a distal end.

### BRIEF DESCRIPTION OF THE DRAWINGS

FIG. 1 is a diagram showing an example of an endoscope system according to an embodiment of the present invention.

FIG. 2 is a diagram showing an example of a treatment tool for an endoscope 20 according to the embodiment of the present invention.

FIG. 3 is a diagram showing a configuration of a grip part 24 of a distal end part 23 in the treatment tool for an endoscope 20.

FIG. 4 is a diagram showing an operation of the grip part 24 of FIG. 3.

FIG. 5 is a diagram showing a configuration of a bendable part 25 and a connecting part 26 of the treatment tool for an endoscope 20.

FIG. 6 is a diagram showing an operation of the bendable part 25 of FIG. 5.

FIG. 7 is a diagram showing an internal configuration of the bendable part 25.

FIG. 8 is a cross-sectional view taken along line a-a of the bendable part 25 shown in FIG. 7.

FIG. 9 is a diagram showing an internal operation of the bendable part 25.

FIG. 10 is a diagram showing a configuration of an operation part 22 of the treatment tool for an endoscope 20.

FIG. 11 is a cross-sectional view of the operation part 22 of the treatment tool for an endoscope 20.

FIG. 12 is a cross-sectional view showing a closed state of the operation part 22 of the treatment tool for an endoscope 20.

FIG. 13 is a diagram showing a first example (part 1) of a treatment method using the treatment tool for an endoscope 20.

FIG. 14 is a diagram showing a first example (part 2) of a treatment method using the treatment tool for an endoscope 20.

FIG. 15 is a diagram showing a first example (part 3) of a treatment method using the treatment tool for an endoscope 20.

FIG. 16 is a diagram showing a first example (part 1) of a treatment method using a combination of the treatment tool for an endoscope 20 and another treatment tool for an endoscope.

FIG. 17 is a diagram showing a first example (part 2) of a treatment method using a combination of the treatment tool for an endoscope 20 and another treatment tool for an endoscope.

FIG. 18 is a diagram showing a first example (part 3) of a treatment method using a combination of the treatment tool for an endoscope 20 and another treatment tool for an endoscope.

FIG. 19 is a diagram showing a state in which the treatment tool for an endoscope 20 is attached to a first treatment tool insertion port 13 of an endoscope operation part 7.

FIG. 20 is a diagram showing the bendable part 25 in an insertion part 21 in a non-bent state.

FIG. 21 is a perspective view showing the bendable part 25 in a bent state.

FIG. 22 is a plan view of the bendable part 25 shown in FIG. 21.

FIG. 23 is a perspective view illustrating a configuration of the distal end part 23 to which an operation wire 27 is connected.

FIG. 24 is a plan view of the distal end part 23 and the operation wire 27 shown in FIG. 23.

FIG. 25 is a perspective view illustrating a configuration of a connecting member 83 that connects the operation wire 27 and link members 81a and 81b.

FIG. 26 is a cross-sectional view of the distal end part 23 and the operation wire 27 taken along a longitudinal direction of the insertion part 21.

FIG. 27 is a view of the distal end part 23 and the operation wire 27 as viewed in a direction of a rotation axis Y of a rotation shaft portion 84.

FIG. 28 is a cross-sectional view taken along line b-b of the distal end part 23 shown in FIG. 27.

FIG. 29 is a diagram showing another example of an operation handle 51.

### DESCRIPTION OF THE PREFERRED EMBODIMENTS

Hereinafter, a treatment tool for an endoscope and an endoscope system according to an embodiment of the present invention will be described with reference to the accompanying drawings.

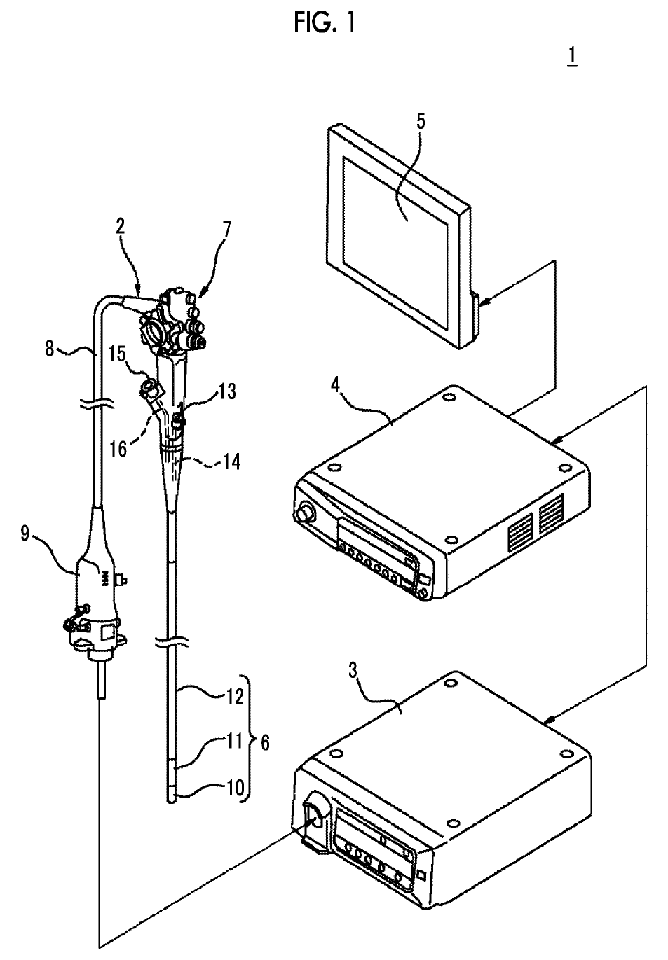

FIG. 1 is a diagram showing an example of an endoscope system according to an embodiment of the present invention. As shown in FIG. 1, an endoscope system 1 comprises an endoscope 2, a light source device 3, and a processor 4. The endoscope 2 has an endoscope insertion part 6 to be inserted into a subject, an endoscope operation part 7 that is connected to the endoscope insertion part 6, and a universal cord 8 that extends from the endoscope operation part 7. The endoscope insertion part 6 is composed of an endoscope distal end part 10, an endoscope bendable part 11 that is connected to the endoscope distal end part 10, and an endoscope connecting part 12 that connects the endoscope bendable part 11 and the endoscope operation part 7.

An imaging apparatus including an imaging element is mounted on the endoscope distal end part 10. The endoscope bendable part 11 is configured to be bendable. The bending of the endoscope bendable part 11 is operated by the endoscope operation part 7. In addition, the endoscope connecting part 12 is configured to be flexible enough to be deformable to follow a shape of an insertion passage in the subject.

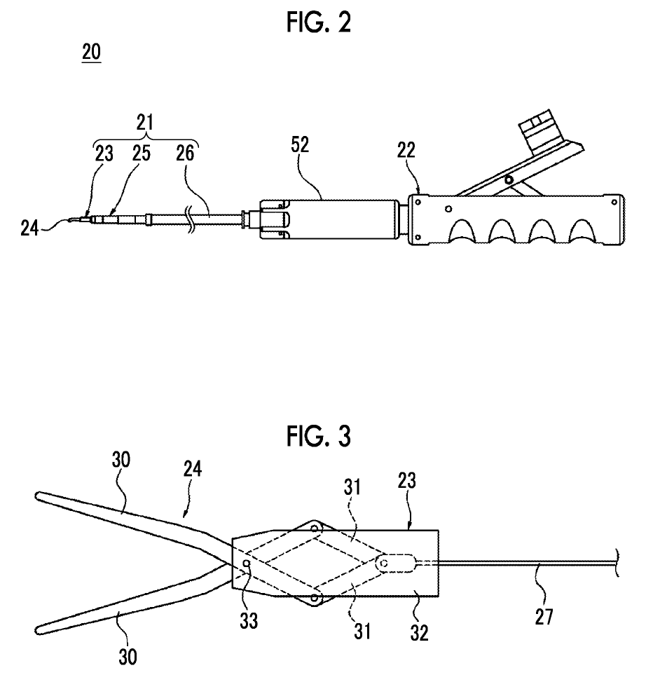

The endoscope operation part 7 is provided with an operation button for operating imaging using the imaging apparatus and an operation knob for operating the bending of the endoscope bendable part 11. In addition, the endoscope operation part 7 is provided with a first treatment tool insertion port 13 into which a treatment tool for an endoscope 20 (see FIG. 2) can be inserted, and a second treatment tool insertion port 15. Inside the endoscope insertion part 6, a first treatment tool channel 14 that reaches the endoscope distal end part 10 from the first treatment tool insertion port 13 and that is open to an end surface of the endoscope distal end part 10, and a second treatment tool channel 16 that reaches the endoscope distal end part 10 from the second treatment tool insertion port 15 and that is open to the end surface of the endoscope distal end part 10 are provided.

A light guide and a cable are provided inside the endoscope insertion part 6, the endoscope operation part 7, and the universal cord 8. A connector 9 is provided at a terminal of the universal cord 8. The endoscope 2 is connected to the light source device 3 and the processor 4 via the connector 9.

Illumination light generated by the light source device 3 is guided to the endoscope distal end part 10 via the light guide and is emitted from the endoscope distal end part 10. In addition, operating power of the imaging element, a control signal for driving the imaging element, and an image signal output from the imaging element are transmitted between the processor 4 and the imaging apparatus via the cable. The processor 4 processes the input image signal to generate image data of an observation site in the subject, displays the generated image data on a monitor 5, and records the generated image data in a storage unit.

FIG. 2 is a view showing an example of the treatment tool for an endoscope 20 according to the embodiment of the present invention. As shown in FIG. 2, the treatment tool for an endoscope 20 comprises an insertion part 21, an operation part 22 of the treatment tool, and a fixing part 52. The insertion part 21 is a portion that can be inserted through the first treatment tool channel 14 (see FIG. 1). The operation part 22 is a portion disposed on a base end side of the insertion part 21. The fixing part 52 is a portion that is attached to and fixed to the endoscope operation part 7.

The insertion part 21 includes a distal end part 23 having a grip part 24 that can be opened and closed by the operation of the operation part 22, a bendable part 25 that is provided adjacent to the operation part 22 side of the distal end part 23, and a connecting part 26 that connects the bendable part 25 and the operation part 22. The grip part 24 is an example of a “treatment part” of the present invention. In a case where the insertion part 21 is inserted through the first treatment tool channel 14, the connecting part 26 is housed in the first treatment tool channel 14. Meanwhile, in a case where the insertion part 21 is inserted through the first treatment tool channel 14, the distal end part 23 and the bendable part 25 protrude from the end surface of the endoscope distal end part 10 (see FIG. 1). As with the endoscope connecting part 12, the connecting part 26 housed in the first treatment tool channel 14 is configured to be flexible enough to be deformable to follow the shape of the insertion passage in the subject.

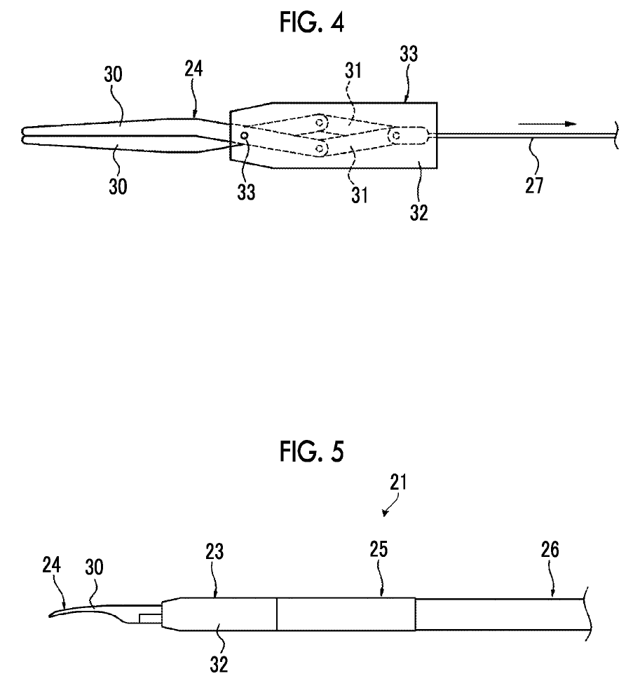

FIG. 3 is a diagram showing a configuration of the grip part 24 of the distal end part 23 in the treatment tool for an endoscope 20. FIG. 4 is a diagram showing an operation of the grip part 24 of FIG. 3.

As shown in FIG. 3, the grip part 24 has a pair of grip claws 30 and a pair of link members 31. The distal end part 23 has a support 32 that rotationally supports the pair of grip claws 30. The support 32 is an example of a “housing” of the present invention. The pair of grip claws 30 are disposed to intersect each other, and a pin 33 is provided to penetrate an intersecting portion of the pair of grip claws 30. The pin 33 is fixed to the support 32. The pair of grip claws 30 are supported by the support 32 so as to be movable rotationally about the pin 33 as a rotational movement shaft.

A distal end part of the link member 31 is rotationally connected to a base end part of the grip claw 30. The operation wire 27 is connected to a base end part of the link member 31. The operation wire 27 reaches the operation part 22 from the distal end part 23 through the bendable part 25 and the connecting part 26, and is pulled to the operation part 22 side or pushed out to the distal end part 23 side in response to an operation of the operation part 22.

FIG. 3 shows a state where the operation wire 27 is pushed out to the distal end part 23 side, and distal end parts of the pair of grip claws 30 are open. On the other hand, in a case where the operation wire 27 is pulled to the operation part 22 side, the distal end parts of the pair of grip claws 30 are closed as shown in FIG. 4. A treatment site of a living body is gripped by the distal end parts of the pair of closed grip claws 30.

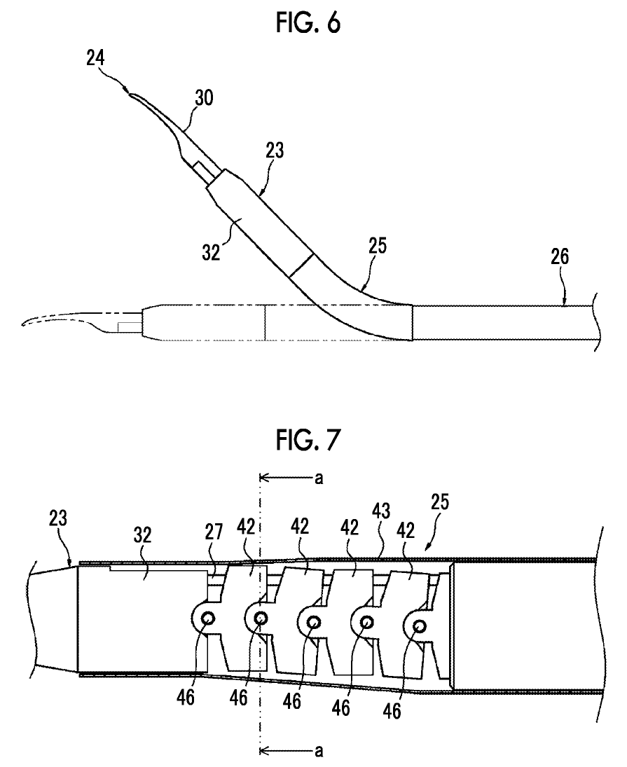

FIG. 5 is a diagram showing a configuration of the bendable part 25 and the connecting part 26 of the treatment tool for an endoscope 20. FIG. 6 is a diagram showing an operation of the bendable part 25 of FIG. 5.

As shown in FIG. 5, in the insertion part 21 of the treatment tool for an endoscope 20, the bendable part 25 is provided on the operation part 22 side of the distal end part 23 having the grip part 24, and the connecting part 26 is provided on the operation part 22 side of the bendable part 25. The connecting part 26 has flexibility and also has stiffness that allows translational and rotational power to be transmitted from the operation part 22 side to the bendable part 25 side. The connecting part 26 may be configured, for example, such that an outer periphery of a spiral tube, which is formed by spirally winding a strip material made of metal, is covered with a net-like tube formed by braiding a wire made of metal and an outer periphery of the net-like tube is covered with an outer coat made of resin.

As shown in FIG. 6, the bendable part 25 operated to be bent by the operation part 22 is bendable in a direction substantially perpendicular to a plane including an opening and closing direction of the pair of grip claws 30. A bending operation plane including a bending operation direction of the bendable part 25 is in a direction substantially perpendicular to an opening and closing operation plane of the pair of grip claws 30.

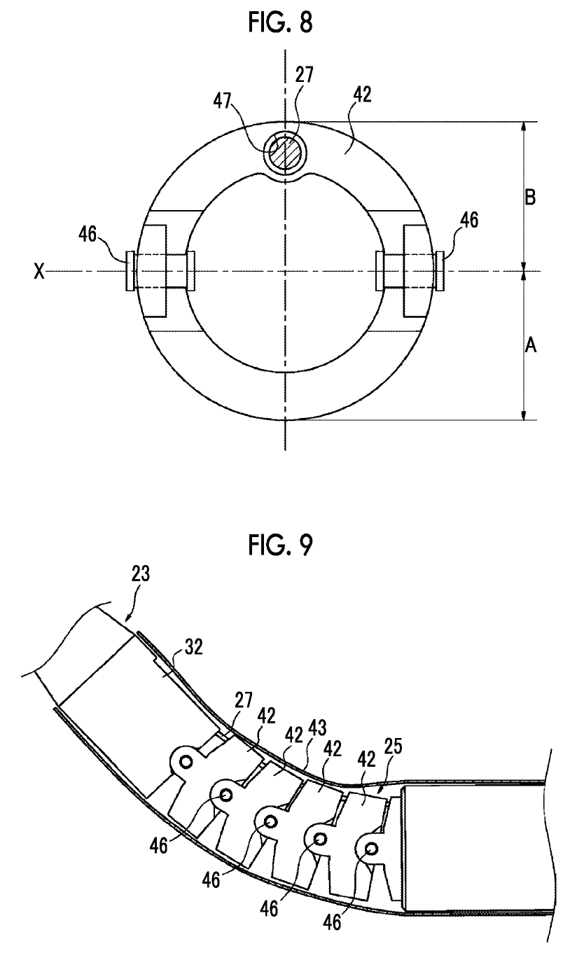

FIG. 7 is a diagram showing an internal configuration of the bendable part 25. FIG. 8 is a cross-sectional view taken along line a-a of the bendable part 25 shown in FIG. 7. FIG. 9 is a diagram showing an internal operation of the bendable part 25.

As shown in FIG. 7, the bendable part 25 has a plurality of bending pieces 42 and an outer coat 43 made of resin. The plurality of bending pieces 42 are arranged in a longitudinal direction of the insertion part 21 including the bendable part 25. Two bending pieces 42 adjacent to each other are connected to each other via a pair of pins 46 disposed to face each other in a radial direction of the bendable part 25.

As shown in FIG. 8, the pair of pins 46 are disposed on an axis X. The axis X is a direction substantially parallel to the opening and closing direction of the pair of grip claws 30. The adjacent bending pieces 42 connected by the pair of pins 46 are rotationally movable about the axis X as a rotational movement axis.

As shown in FIG. 9, the bendable part 25 is bent along a direction (bending operation plane) substantially perpendicular to the opening and closing direction (direction of the axis X) of the pair of grip claws 30 by adding up rotational movements of the plurality of bending pieces 42 with the axis X as the rotational movement axis.

The bendable part 25 is bent by the operation wire 27 for opening and closing the pair of grip claws 30. As shown in FIG. 8, each of the plurality of bending pieces 42 has a wire guide 47 that holds the operation wire 27 in a pushable and pullable manner. In a case where the bending piece 42 is divided into a first side A and a second side B, which is an opposite side, with the axis X as a boundary, the wire guide 47 is provided on one side, which is the second side B. Therefore, as the operation wire 27 is pulled to the operation part 22 side, the bendable part 25 is bent such that the first side A is positioned outside the curve and the second side B is positioned inside the curve.

As described above, in the treatment tool for an endoscope 20, the closing operation of the grip part 24 and the bending operation of the bendable part 25 are performed by pulling one operation wire 27. This makes it easier to operate the operation part 22. Here, in a case where the operation wire 27 is pulled to the operation part 22 side, the grip part 24 is first closed, and the bendable part 25 is bent in a state where the grip part 24 is closed. An operation sequence of the closing operation of the grip part 24 and the bending operation of the bendable part 25 can be set based on a magnitude relationship between an operation resistance in a case of closing the grip part 24 and an operation resistance in a case of bending the bendable part 25. For example, in a case where the operation resistance of the bendable part 25 is relatively large, the closing operation of the grip part 24 is performed first, and the bending operation of the bendable part 25 is performed later.

The operation resistance in a case where the grip part 24 is closed includes friction at the intersecting portion of the pair of grip claws 30 and friction at a connecting portion between the grip claw 30 and the link member 31. The operation resistance in a case where the bendable part 25 is bent includes friction at a connecting portion between the two adjacent bending pieces 42. In addition, the outer coat 43 of the bendable part 25 is an elastic member that linearly extends the bendable part 25. Therefore, the operation resistance in a case where the bendable part 25 is bent includes the elasticity of the outer coat 43. In addition, the operation wire 27 is also an elastic member that linearly extends the bendable part 25. Therefore, the operation resistance in a case where the bendable part 25 is bent includes the elasticity of the operation wire 27. The elastic member that extends the bendable part 25 linearly is not limited to the outer coat 43, the operation wire 27, and the like, and may be a wire spring, a leaf spring, or the like.

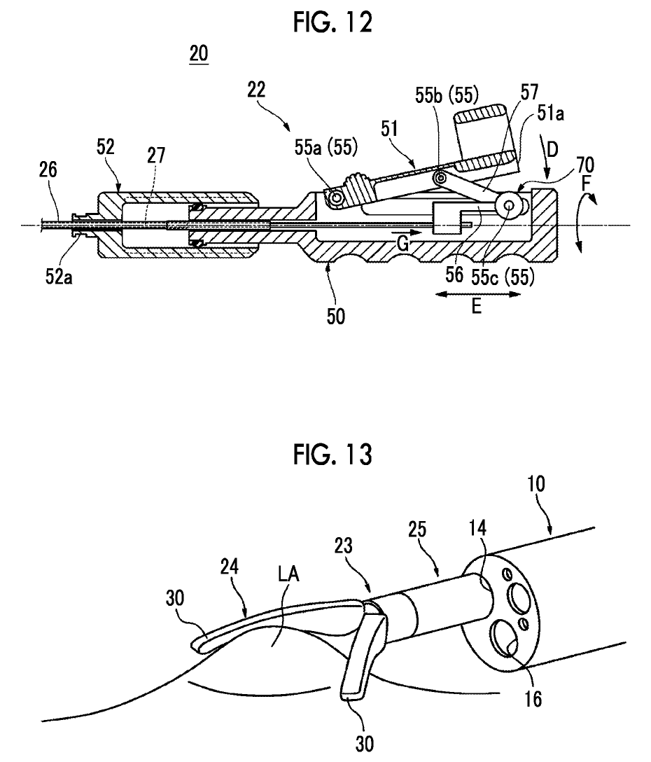

As described above, in the bendable part 25 that is bent by the pulling of the operation wire 27, as the outer coat 43 is made of an elastic member, an elastic force in a direction returning from a bent state to a linear state is generated by the outer coat 43. In addition, the elastic force increases according to the amount of bending of the bendable part 25. A friction adjustment mechanism 70 (described below in FIG. 12) is provided which can respond to a change in the elastic force, increase a frictional force according to the bending amount (angle) of the bendable part 25, and maintain the bent state of the bendable part 25 in a stable condition.

As the amount of movement by which the operation wire 27 is pulled increases, a force pulling in a direction of restoring the operation wire 27 (a linearly extending force) increases. In the present embodiment, the friction adjustment mechanism 70 to be described below, which can respond to a change in the force (elastic restoring force) pulling in the direction of restoring the operation wire 27, can increase a frictional force to correspond to the amount of pulling of the operation wire 27, and can maintain a pulled state in a stable condition.

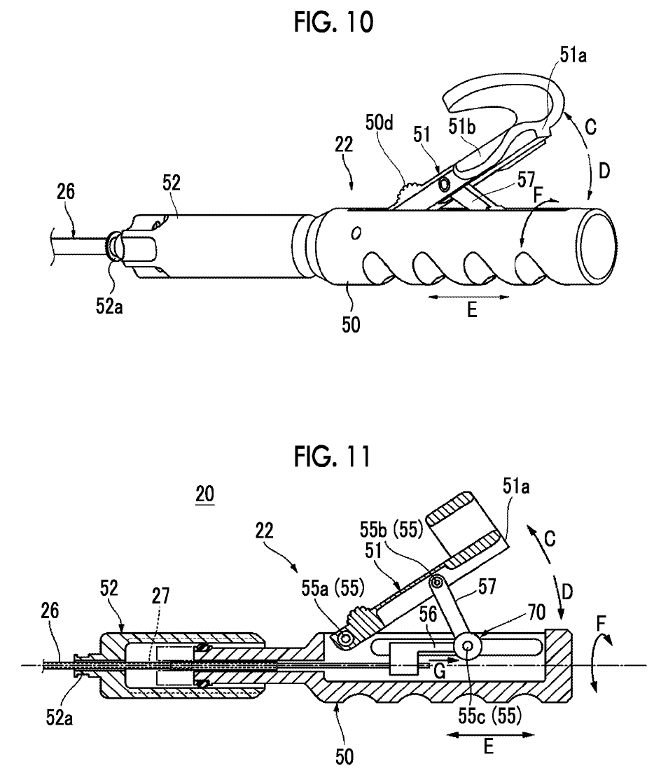

FIG. 10 is a diagram showing a configuration of the operation part 22 of the treatment tool for an endoscope 20. FIG. 11 is a cross-sectional view of the operation part 22 of the treatment tool for an endoscope 20.

As shown in FIG. 10, the operation part 22 includes an operation part main body 50, an operation handle 51, and a fixing part 52. The operation part main body 50 functions as means for inputting a forward and backward rotation operation of moving the grip part 24 (see FIG. 3) forward and backward and rotating the grip part 24. The operation handle 51 functions as means for inputting an opening and closing bending operation of opening and closing the grip part 24 and bending the bendable part 25 (see FIG. 2). The fixing part 52 is an attachment portion that is attachably and detachably attached to the endoscope operation part 7 (see FIG. 1).

The fixing part 52 has a connection metal fitting 52a. The connection metal fitting 52a is connected to a base provided at the first treatment tool insertion port 13 (see FIG. 1) of the endoscope operation part 7. The operation part 22 is supported by the endoscope operation part 7 in a state in which the connection metal fitting 52a is connected to the base.

The operation part main body 50 is formed in a rod shape and is operable in a central axis direction indicated by an arrow E and in a rotation direction around a central axis indicated by an arrow F. The operation part main body 50 is supported by the fixing part 52. The connecting part 26 of the insertion part 21 is connected to the operation part main body 50 through the fixing part 52. The connecting part 26 is moved forward and backward along a longitudinal axis of the connecting part 26 in response to the operation of the operation part main body 50 in the direction of the arrow E. In addition, the connecting part 26 is rotated about the longitudinal axis of the connecting part 26 in response to the operation of the operation part main body 50 in the direction of the arrow F. The forward and backward movement and rotation of the connecting part 26 are transmitted to the grip part 24, and the grip part 24 is also moved forward and backward and is rotated integrally with the connecting part 26.

The operation handle 51 is swingably supported by the operation part main body 50. For example, as shown in FIG. 11, the operation handle 51 is supported by a rotational movement support shaft part 55a (55), and can swing in an opening direction C in which a free end part 51a moves away from the operation part main body 50 and in a closing direction D approaching the operation part main body 50. The operation handle 51 is supported to move on a fan-shaped operation movement surface centered on the rotational movement support shaft part 55a.

FIG. 12 is a cross-sectional view showing a closed state of the operation part 22 of the treatment tool for an endoscope 20. The closed state of the operation part 22 is a state in which the operation handle 51 is operated in the closing direction D. Meanwhile, the open state of the operation part 22 is a state (FIG. 11) in a case where the operation handle 51 is operated in the opening direction C.

The operation handle 51 pulls (direction of an arrow G) the operation wire 27 in a case of swinging in the closing direction D and pushes (direction opposite to the arrow G) the operation wire 27 in a case of swinging in the opening direction C. The operation wire 27 is fixed to a wire holding part 56 that is provided to be slidably movable along its axis in the operation part main body 50. The wire holding part 56 is connected to the operation handle 51 via a link member 57 that is rotationally connected to the rotational movement support shaft part 55c (55). The link member 57 and the operation handle 51 are rotationally connected to each other via the rotational movement support shaft part 55b (55).

As described above, as the amount of pulling (the amount of movement in the direction of the arrow G) of the operation wire 27 increases, a force (elastic restoring force) pulling in a direction of restoring the wire holding part 56 increases. In addition, in a case where a lesion part is gripped and lifted, the restoring force increases with the lifting amount (the amount of pulling). Therefore, a restoring force acting on the operation handle 51 increases as the amount of pulling increases. In order to respond to a change in the restoring force, the friction adjustment mechanism 70 that increases a frictional force for locking the wire holding part 56 to correspond to the pulling amount of the operation wire 27 that is interlocked with an operation of the operation handle 51 is provided at the rotational movement support shaft part 55c.

The operation wire 27 is pushed out to the distal end part 23 side in response to the swinging of the operation handle 51 in the opening direction C and is pulled to the operation part 22 side in response to the swinging of the operation handle 51 in the closing direction D. In this case, as the operation handle 51 swings in the closing direction D, the grip part 24 is closed and the bendable part 25 is bent.

As the operation handle 51 has the friction adjustment mechanism 70 that maintains its operation state, the operation handle 51 is configured to be capable of maintaining a bending angle of the bendable part 25 at any one of angles that are equal to or smaller than a maximum bending angle. The friction adjustment mechanism 70 increases a frictional force for locking the wire holding part 56 to correspond to an increase in the pulling amount of the operation wire 27 caused by the wire holding part 56 that is interlocked with the swinging of the operation handle 51. Therefore, even in a case where the pulling amount of the operation wire 27 is increased and the wire restoring force is increased, the operation handle 51 is prevented from returning. Accordingly, a hand can be released from the operation handle 51 regardless of an operation position of the operation handle 51, and further, an operation force required for the rotational movement is small, so that a stable operation is possible.

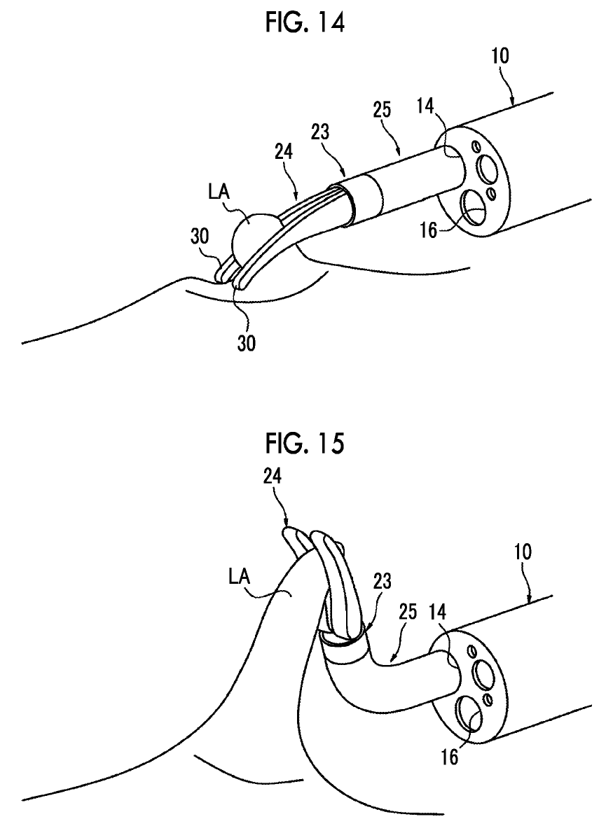

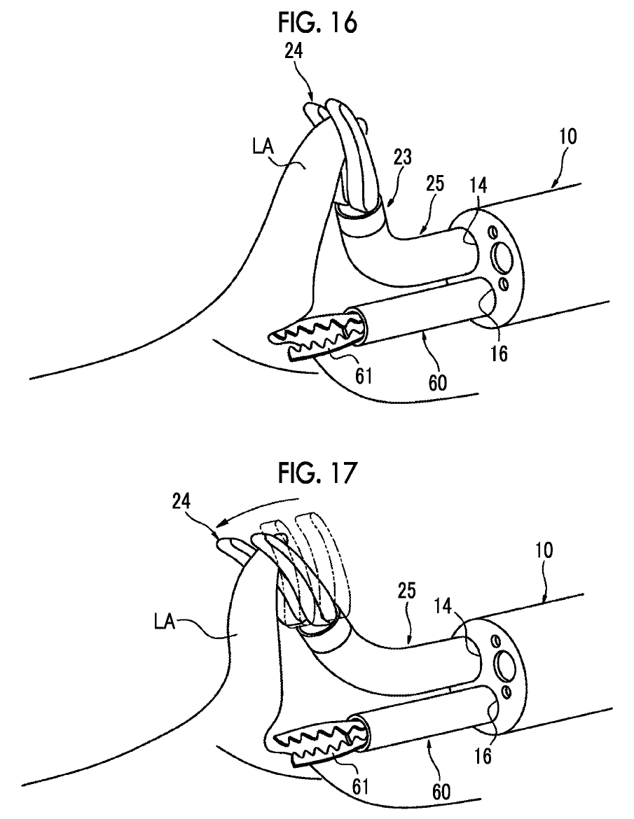

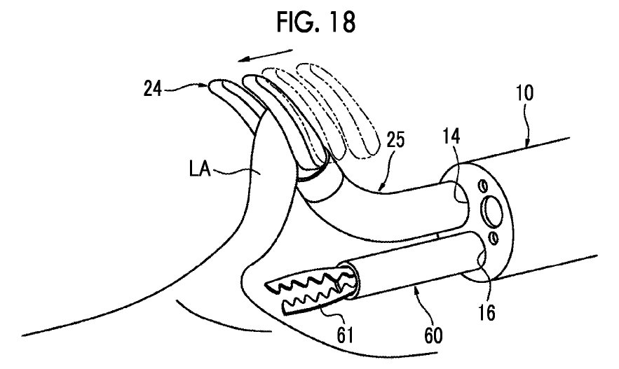

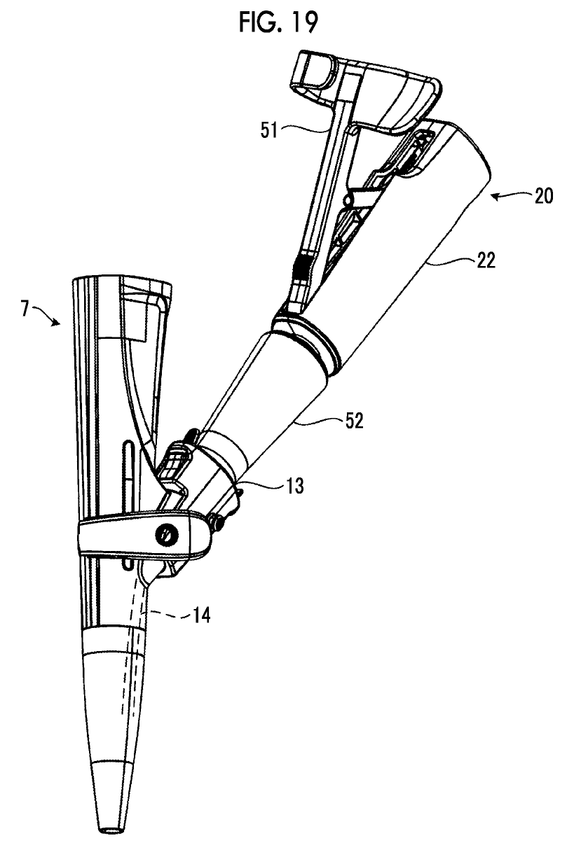

A first example of a treatment method using the treatment tool for an endoscope 20 will be described with reference to FIGS. 13 to 18. In this example, a treatment method of endoscopic submucosal dissection (ESD) will be described. FIG. 13 is a diagram showing a first example (part 1) of a treatment method using the treatment tool for an endoscope 20. FIG. 14 is a diagram showing a first example (part 2) of a treatment method using the treatment tool for an endoscope 20. FIG. 15 is a diagram showing a first example (part 3) of a treatment method using the treatment tool for an endoscope 20. In addition, FIG. 16 is a diagram showing a first example (part 1) of a treatment method using a combination of the treatment tool for an endoscope 20 and another treatment tool for an endoscope. FIG. 17 is a diagram showing a first example (part 2) of a treatment method using a combination of the treatment tool for an endoscope 20 and another treatment tool for an endoscope. FIG. 18 is a diagram showing a first example (part 3) of a treatment method using a combination of the treatment tool for an endoscope 20 and another treatment tool for an endoscope.

The operation wire 27 is pulled to the operation part 22 side in response to the swinging of the operation handle 51 in the closing direction D. In addition, another treatment tool for an endoscope used in combination with the treatment tool for an endoscope 20 is an incision tool and is high-frequency forceps 60 having a pair of openable and closable claws 61 at a distal end part (see FIG. 16). The pair of claws 61 are opened and closed by operating an operation part of the high-frequency forceps 60. In a state where the pair of claws 61 are closed and a living body tissue is gripped by the pair of claws 61, a high-frequency current flows between the pair of claws 61 and a return electrode plate via the living body tissue, or a high-frequency current flows between the pair of claws 61, thereby cauterizing and incising the living body tissue.

As shown in FIG. 13, the endoscope distal end part 10 of the endoscope 2 inserted into the body is disposed on a side of a lesion part LA of a mucous membrane layer. The treatment tool for an endoscope 20 is inserted through the first treatment tool channel 14 of the endoscope 2, and the distal end part 23 and the bendable part 25 of the treatment tool for an endoscope 20 protrude from the end surface of the endoscope distal end part 10. Then, the lesion part LA is gripped by the grip part 24 of the distal end part 23 through the operation of the operation part 22 of the treatment tool for an endoscope 20.

In a case where the lesion part LA is gripped by the grip part 24, first, the operation handle 51 (see FIG. 10) of the operation part 22 is operated in the opening direction C. As shown in FIG. 13, by operating the operation handle 51 in the opening direction C, the operation wire 27 is pushed to the distal end part 23 side, and, by pushing the operation wire 27, the bendable part 25 is linearly extended and laid along the longitudinal axis of the connecting part 26. In addition, as the operation wire 27 is pushed out, the pair of grip claws 30 of the grip part 24 are opened. Then, the lesion part LA is disposed between the pair of grip claws 30 by appropriately pushing and pulling the operation part main body 50.

Next, the operation handle 51 is operated in the closing direction D in a state where the lesion part LA is disposed between the pair of grip claws 30. Accordingly, the operation wire 27 is pulled to the operation part 22 side. As the operation wire 27 is pulled, first, as shown in FIG. 14, the pair of grip claws 30 are closed and the lesion part LA is gripped by the grip part 24. Then, after the lesion part LA is gripped by the grip part 24, the bendable part 25 is bent as shown in FIG. 15. Accordingly, the grip part 24 is erected from a state of being laid along the longitudinal axis of the connecting part 26, and the lesion part LA gripped by the grip part 24 is lifted.

In a state where the lesion part LA is being lifted, the high-frequency forceps 60 inserted through the second treatment tool channel 16 of the endoscope 2 protrude from an edge surface of the endoscope distal end part 10 as shown in FIG. 16. The pair of claws 61 of the high-frequency forceps 60 are disposed at a lower part of the lesion part LA, and the lower part of the lesion part LA is incised by the pair of claws 61. As the incision proceeds, the lifted lesion part LA may be released once, re-gripped, and then lifted. In a case where the lesion part LA that has been incised once is lifted, the lower part can be exposed for easy viewing. The incision proceeds as the high-frequency forceps 60 is pushed and pulled as appropriate, and the lesion part LA including a submucosal layer is gradually peeled off.

In this way, the lesion part LA can be gripped from the side of the lesion part LA, and the gripped lesion part LA can be lifted by operating the operation handle 51 of the treatment tool for an endoscope 20, so that the operation is simple. In addition, by lifting the lesion part LA, the lower part of the lesion part LA can be exposed for easy viewing, and treatment for the lower part of the lifted lesion part LA can be performed safely, reliably, and easily.

In a case of incision, the operation part main body 50 of the operation part 22 may be pushed and pulled in the direction of the arrow E of FIG. 10 and/or the operation part main body 50 may be rotated in the direction of the arrow F of FIG. 10. As described above, the connecting part 26 has stiffness that allows translational and rotational power to be transmitted from the operation part 22 side to the bendable part 25 side, and pushing, pulling, and rotation of the operation part main body 50 are transmitted to the bendable part 25 via the connecting part 26.

FIG. 17 shows a movement of the grip part 24 in a case where the operation part main body 50 is rotated. The connecting part 26 is rotated about the longitudinal axis of the connecting part 26 in response to the rotation of the operation part main body 50. In a state in which the bendable part 25 is bent, the grip part 24 is rotated while maintaining a state of being erected with respect to the longitudinal axis of the connecting part 26. As the grip part 24 is rotated, the lesion part LA gripped by the grip part 24 is swung around the longitudinal axis of the connecting part 26.

FIG. 18 shows a movement of the grip part 24 in a case where the operation part main body 50 is pushed and pulled. The connecting part 26 is moved forward and backward in an axial direction of the longitudinal axis of the connecting part 26 in response to the pushing and pulling of the operation part main body 50. In a state in which the bendable part 25 is bent, the grip part 24 is moved forward and backward while maintaining a state of being erected with respect to the longitudinal axis of the connecting part 26. As the grip part 24 is moved forward and backward, the lesion part LA gripped by the grip part 24 is pushed and pulled in the axial direction of the longitudinal axis of the connecting part 26.

By swinging and/or pushing and pulling the lesion part LA as appropriate, for example, an incised wound can be widened. Accordingly, treatment for the lower part of the lesion part LA can be performed more easily.

FIG. 19 is a diagram showing a state in which the treatment tool for an endoscope 20 is attached to the first treatment tool insertion port 13 of the endoscope operation part 7. As described above, the treatment tool for an endoscope 20 is attached to the endoscope operation part 7 by connecting the connection metal fitting 52a of the fixing part 52 to the base of the first treatment tool insertion port 13 of the endoscope operation part 7. In the treatment tool for an endoscope 20 attached to the endoscope operation part 7, the insertion part 21 consisting of the distal end part 23, the bendable part 25, and the connecting part 26 is inserted into the first treatment tool channel 14 that reaches the endoscope distal end part 10 from the first treatment tool insertion port 13.

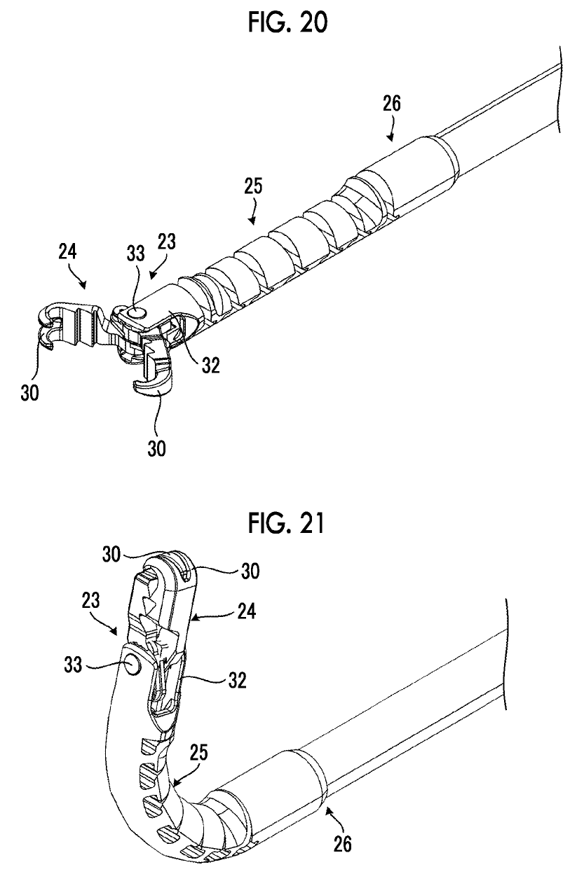

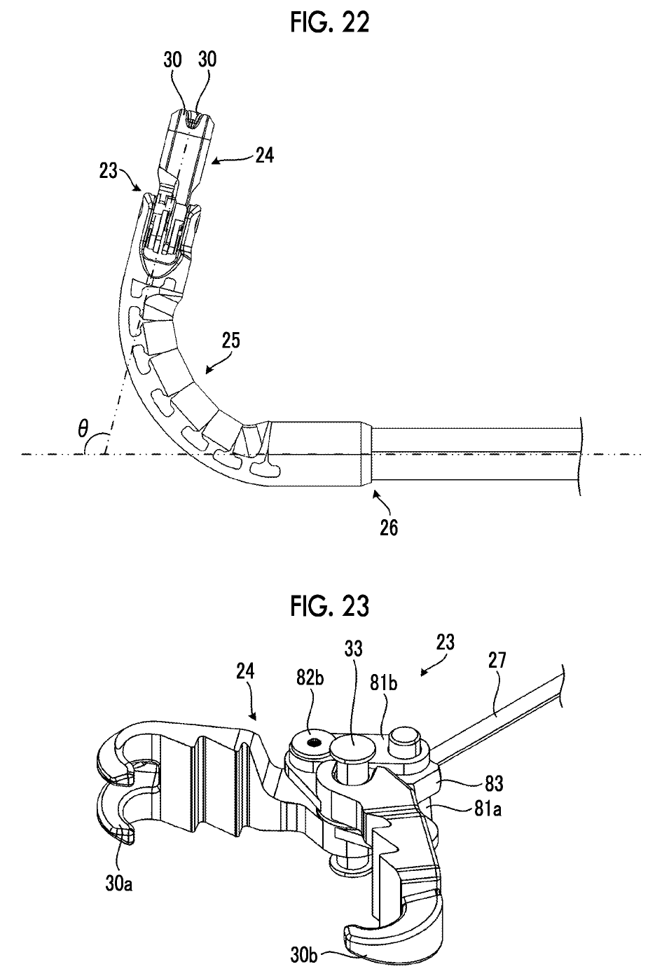

The bent state of the bendable part 25 in the insertion part 21 of the treatment tool for an endoscope 20 will be described with reference to FIGS. 20 to 22. FIG. 20 is a diagram showing the bendable part 25 in the insertion part 21 in a non-bent state. FIG. 21 is a perspective view showing the bendable part 25 in a bent state. FIG. 22 is a plan view of the bendable part 25 shown in FIG. 21.

As shown in FIG. 20, the non-bent state of the bendable part 25 means a state in which the bendable part 25 is straight with respect to an end part of the connecting part 26 on the bendable part 25 side. As described above, the bendable part 25 is bent by the operation of the operation wire 27 that reaches the operation part 22 from the distal end part 23 through the bendable part 25 and the connecting part 26. In addition, the pair of grip claws 30 at the distal end part 23 are opened and closed by the operation of the operation wire 27.

For example, in a case where the operation wire 27 is pushed out to the distal end part 23 side, as shown in FIG. 20, the bendable part 25 is brought into a non-bent state where the bendable part25 is linearly extended and laid along the longitudinal axis of the connecting part 26, and the pair of grip claws 30 of the grip part 24 are in an open state.

On the other hand, in a case where the operation wire 27 is pulled to the operation part 22 side, as shown in FIGS. 21 and 22, the pair of grip claws 30 of the grip part 24 are brought into a closed state, and the bendable part 25 is bent so as to bulge and is brought into an upwardly bent state with respect to the longitudinal axis of the connecting part 26. In this case, in a case where the operation wire 27 is pulled to the operation part 22 side, first, the pair of grip claws 30 are brought into a closed state, and, after the pair of grip claws 30 are closed, the bendable part 25 is brought into a bent state.

As shown in FIG. 22, a maximum bending angle θ of the bendable part 25 with respect to the longitudinal axis of the connecting part 26 is configured to be greater than 90 degrees. The expression “maximum bending angle” refers to an angle of a direction of the distal end of the bendable part 25 in a case where the bendable part 25 is maximally bent relative to a direction of the distal end of the bendable part 25 in a case where the bendable part 25 is in a non-bent state. The expression “maximally bent” refers to a state in which the bendable part 25 is bent to the greatest extent within a range that can be operated by operating the operation part 22. The maximum bending angle θ is preferably, for example, 100 degrees to 180 degrees.

By setting the maximum bending angle of the bendable part 25 to be greater than 90 degrees in this way, it is possible to continuously pull the lesion part LA gripped by the grip claws 30 over a wide range. Therefore, for example, even in a case where the lesion part LA to be incised is a large part, or even in a case where the incision process of the lesion part LA has progressed to a certain extent, the number of times the lesion part LA needs to be re-gripped by the grip claws 30 can be reduced. As a result, the lower part of the lesion part LA can be exposed for easy viewing, and treatment for the lower part of the lesion part LA can be performed safely, reliably, and easily.

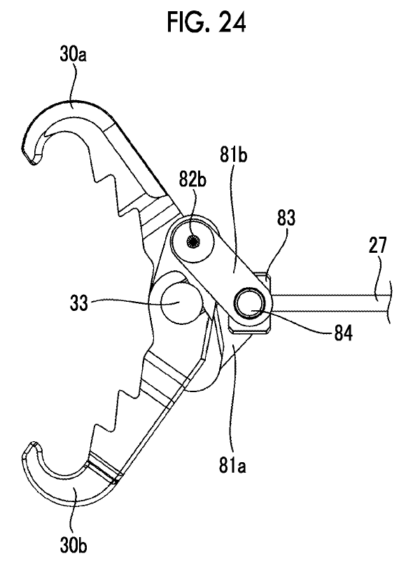

A configuration of the distal end part 23 to which the operation wire 27 is connected will be described with reference to FIGS. 23 to 28. FIG. 23 is a perspective view illustrating the configuration of the distal end part 23 to which the operation wire 27 is connected. FIG. 24 is a plan view of the distal end part 23 and the operation wire 27 shown in FIG. 23. In FIGS. 23 and 24, the support 32 of the distal end part 23 is omitted for ease of viewing.

As shown in FIGS. 23 and 24, the operation wire 27 is connected to the grip part 24 (treatment part) of the distal end part 23. The grip part 24 includes grip claws 30a and 30b, link members 81a and 81b that are rotationally connected to a base end side of the grip claws 30a and 30b, and a connecting member 83 that is rotationally connected to a base end side of the link members 81a and 81b. The grip claws 30a and 30b are examples of a “distal end operation part” of the present invention.

The pair of grip claws 30a and 30b are disposed to intersect with each other, as with the grip claws 30 described in FIG. 3, and are provided to be movable rotationally about a pin 33 that penetrates an intersecting portion therebetween as a rotational movement shaft.

The link member 81a is connected to the grip claw 30a. The link member 81b is connected to the grip claw 30b. The link member 81b and the grip claw 30b are disposed such that end parts thereof intersect with each other, and are connected to be movable rotationally about a pin 82b provided to penetrate through an intersecting portion therebetween as a rotational movement shaft. Similarly, the link member 81a and the grip claw 30a are disposed such that end parts thereof intersect with each other, and are connected to be movable rotationally about a pin (not shown) provided to penetrate through an intersecting portion therebetween as a rotational movement shaft.

An end part of the link member 81a on a side opposite to an end part connected to the grip claw 30a is connected to the connecting member 83. Similarly, an end part of the link member 81b on a side opposite to an end part connected to the grip claw 30b is connected to the connecting member 83. The link member 81a is connected to a rotation shaft portion 84 of the connecting member 83. Similarly, the link member 81b is connected to the rotation shaft portion 84 of the connecting member 83.

The operation wire 27 is fixed to the connecting member 83 of the grip part 24. The connecting member 83 is a sleeve member that connects the operation wire 27 and the link members 81a and 81b. In a longitudinal axis direction of the insertion part 21, at least a part of the operation wire 27 overlaps the link members 81a and 81b connected to the rotation shaft portion 84 and is fixed to the connecting member 83. As a method of fixing the connecting member 83 and the operation wire 27, laser welding, soldering, brazing, caulking, or the like can be used, but soldering or brazing may cause solder or brazing material to permeate a base end side of a wire, and, in this case, the wire in the permeated area may harden and hinder the bending of the bendable part 25. Therefore, a method of welding an area controlled by laser welding is suitable. In addition, by avoiding a rear end surface (end surface on the side of the operation part 22) of the connecting member 83 in the laser welding area, the influence of changes in the stiffness of the wire due to welding on bending can be minimized. For example, a distal end surface of the connecting member 83 (end surface on the side of the grip part 24) and a distal end surface of the operation wire 27 are aligned with each other, and the laser welding is performed on these distal end surfaces, so that it is possible to weld a limited area including the distal end surfaces.

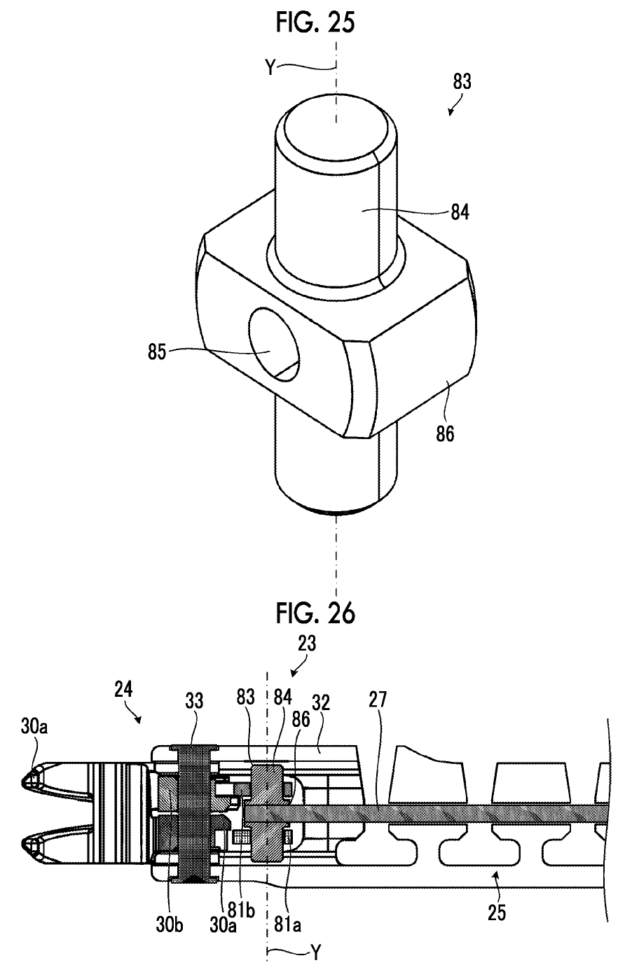

FIG. 25 is a perspective view illustrating a configuration of the connecting member 83 that connects the operation wire 27 and the link members 81a and 81b. The connecting member 83 has a rotation shaft portion 84 that rotationally connects the link members 81a and 81b and the connecting member 83, and an insertion hole 85 through which the operation wire 27 is insertable. The rotation shaft portion 84 is configured, for example, in a columnar shape. The insertion hole 85 is formed to intersect with a rotation axis Y of the rotation shaft portion 84. The insertion hole 85 is formed to penetrate the rotation shaft portion 84 in a direction orthogonal to a direction of the rotation axis Y of the rotation shaft portion 84. The operation wire 27 inserted through the insertion hole 85 is fixed to an inner surface of the insertion hole 85. The expression “rotation axis Y of the rotation shaft portion 84” is an imaginary rotation axis (straight line).

At a central portion of the rotation shaft portion 84 in the direction of the rotation axis Y, for example, a rectangular extending portion 86 that extends in an insertion direction of the operation wire 27 is provided. The insertion hole 85 of the connecting member 83 penetrates the rotation shaft portion 84 and is formed to penetrate the extending portion 86. Therefore, in the longitudinal axis direction of the insertion part 21, a length of the insertion hole 85 is longer than a length (diameter) of the rotation shaft portion 84. Accordingly, in the longitudinal axis direction of the insertion part 21, a length of a portion where the inner surface of the insertion hole 85 and the operation wire 27 are in contact with each other is longer than the length (diameter) of the rotation shaft portion 84. For example, the connecting member 83 is integrally formed to include the rotation shaft portion 84, the extending portion 86, and the insertion hole 85.

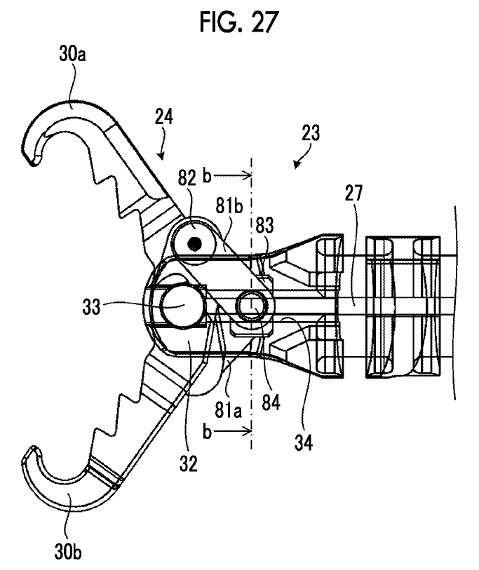

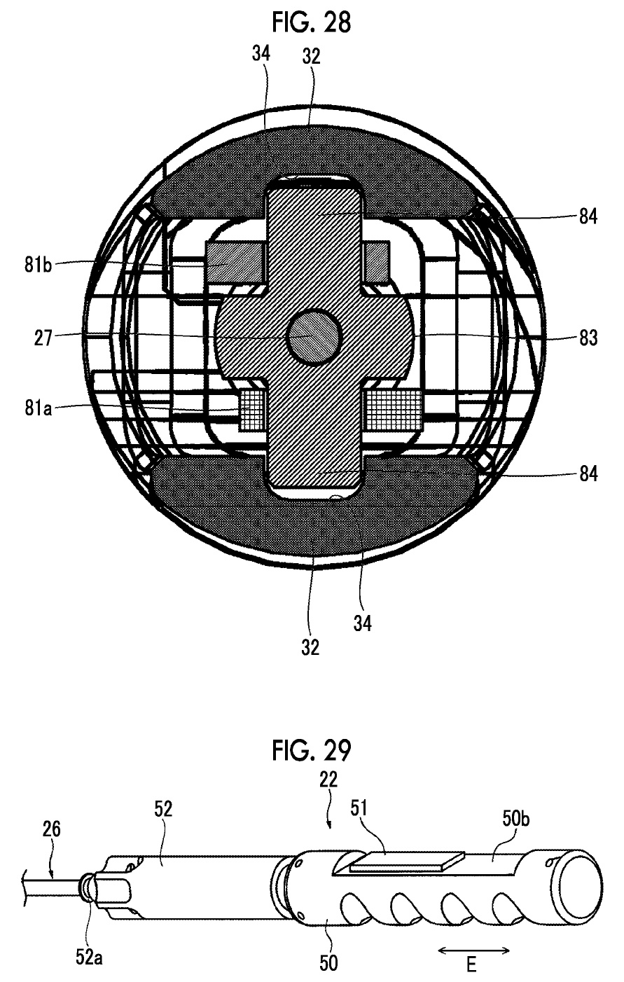

FIG. 26 is a cross-sectional view of the distal end part 23 and the operation wire 27 taken along the longitudinal direction of the insertion part 21. FIG. 27 is a view of the distal end part 23 and the operation wire 27 as viewed in the direction of the rotation axis Y of the rotation shaft portion 84. FIG. 28 is a cross-sectional view taken along line b-b of the distal end part 23 shown in FIG. 27. In FIGS. 26 to 28, the support 32 of the distal end part 23 is shown.

As shown in FIG. 26, the link member 81a and the link member 81b are each connected to the rotation shaft portion 84 of the connecting member 83. The link member 81a and the link member 81b are connected to one side and the other side (an upper side and a lower side in FIG. 26) of the rotation shaft portion 84 with the extending portion 86 formed on the rotation shaft portion 84 interposed therebetween. An end part of the operation wire 27 inserted through the insertion hole 85 formed in the rotation shaft portion 84 and the extending portion 86 overlaps the link members 81a and 81b and is fixed to the connecting member 83.

As shown in FIGS. 26 and 27, the pin 33 that is provided to penetrate the intersecting portion of the pair of grip claws 30a and 30b is fixed to the support 32 that houses the connecting member 83. The support 32 is provided, for example, to cover the connecting member 83 from both sides in the direction of the rotation axis Y of the rotation shaft portion 84. The pin 33 rotationally fixes the support 32, the link members 81a and 81b, and the grip claws 30a and 30b.

As shown in FIGS. 27 and 28, a guide groove 34 for guiding the movement of the connecting member 83 is formed in the support 32. The guide groove 34 is formed in the support 32 along the longitudinal axis direction of the insertion part 21. Both ends in the direction of the rotation axis Y of the rotation shaft portion 84 of the connecting member 83 are engaged with the guide groove 34. For example, in a case where the operation wire 27 is pulled to the operation part 22 side, the rotation shaft portion 84 of the connecting member 83 connected to the operation wire 27 is guided by the guide groove 34 of the support 32 and moves to the operation part 22 side in the longitudinal axis direction of the insertion part 21. Similarly, in a case where the operation wire 27 is pushed out to the distal end part 23 side, the rotation shaft portion 84 of the connecting member 83 is guided by the guide groove 34 and moves to the distal end part 23 side in the longitudinal axis direction of the insertion part 21.

In FIGS. 23 to 28, the grip part 24 of the distal end part 23 of the treatment tool for an endoscope 20 has been described as an example of the “treatment part”, but the present invention is not limited to this. An example of the “treatment part” may be a distal end part of the high-frequency forceps 60 or the like.

As described above, the treatment tool for an endoscope 20 comprises the grip part 24 including the grip claws 30a and 30b, the link members 81a and 81b that are rotationally connected to the base end side of the grip claws 30a and 30b, and the connecting member 83 that is rotationally connected to the base end side of the link members 81a and 81b, and at least one operation wire 27 that is fixed to the connecting member 83 and that operates the opening and closing of the grip claws 30a and 30b, and is disposed such that at least a part of the operation wire 27 fixed to the connecting member 83 and the link members 81a and 81b connected to the connecting member 83 overlap each other in the longitudinal axis direction of the insertion part 21 that is insertable into the body. With this configuration, since the operation wire 27 and the link members 81a and 81b are connected to the connecting member 83 and the connected members are disposed to overlap each other, the length of the grip part 24 in the longitudinal axis direction of the insertion part 21 can be shortened. Accordingly, even in a state where the first treatment tool channel 14 of the endoscope operation part 7 is bent, the insertion part 21 of the treatment tool for an endoscope 20 can be easily inserted through the first treatment tool channel 14. In addition, the grip part 24 extending from the endoscope distal end part 10 can be operated at a position close to the end surface of the endoscope distal end part 10. Therefore, the operability is improved by shortening the grip part 24 of the distal end part 23.

In addition, with the treatment tool for an endoscope 20, the connecting member 83 is provided with the rotation shaft portion 84 that rotationally connects the link members 81a and 81b and the connecting member 83, and the insertion hole 85 through which the operation wire 27 is insertable and which intersects with the rotation axis Y of the rotation shaft portion 84, and the operation wire 27 is fixed to the inner surface of the insertion hole 85. Accordingly, since the operation wire 27 inserted through the insertion hole 85 that intersects with the rotation axis Y of the rotation shaft portion 84 and the link members 81a and 81b are disposed to overlap each other, the length of the grip part 24 in the longitudinal axis direction of the insertion part 21 can be shortened, and the operability is improved.

In addition, with the treatment tool for an endoscope 20, the rotation shaft portion 84 is provided with the extending portion 86 that extends in the insertion direction of the operation wire 27, and the insertion hole 85 through which the operation wire 27 is inserted is provided in the extending portion 86. Therefore, in the longitudinal axis direction of the insertion part 21, the length of the insertion hole 85 can be made longer than the length (diameter) of the rotation shaft portion 84, so that a portion where the inner surface of the insertion hole 85 and the operation wire 27 are in contact with each other can be made longer than the diameter of the rotation shaft portion 84. Accordingly, the fixing strength of the operation wire 27 fixed to the inner surface of the insertion hole 85 can be increased.

In addition, with the treatment tool for an endoscope 20, the support 32 that houses the connecting member 83 is provided with the guide groove 34 for guiding the movement of the rotation shaft portion 84 in the longitudinal axis direction of the insertion part 21. Therefore, in a case where the operation wire 27 is pulled or pushed, the rotation shaft portion 84 can be guided along the guide groove 34 to be moved along the longitudinal axis direction of the insertion part 21. Accordingly, the link members 81a and 81b connected to the rotation shaft portion 84 and the pair of grip claws 30a and 30b connected to the link members 81a and 81b can be operated symmetrically about the longitudinal axis of the insertion part 21. Therefore, the operability of the treatment tool for an endoscope 20 can be improved.

In the operation part 22 of the above embodiment, the operation handle 51 is provided to protrude from the operation part main body 50 and to be swingable, and the wire holding part 56 is moved by the swing, but the present invention is not limited to this configuration. FIG. 29 is a diagram showing another example of the operation handle 51. For example, as shown in FIG. 29, the operation handle 51 may be a member that is provided on a plane portion 50b provided at the operation part main body 50 and that is movable in a direction E along the plane portion 50b. The operation handle 51 is operated, for example, by a thumb of an operator gripping the operation part main body 50.

In this case, the operation handle 51 is connected to the wire holding part 56, and, in a case where the operation handle 51 is moved to a distal end side (an upper side of FIG. 29) of the operation part main body 50, the wire holding part 56 is also moved to the distal end side of the operation part main body 50, and the operation wire 27 is pulled. In addition, in a case where the operation handle 51 is moved to a base end side (a lower side of FIG. 29) of the operation part main body 50, the wire holding part 56 is also moved to the base end side of the operation part main body 50, and the operation wire 27 is loosened. That is, in the configuration shown in FIG. 29, through an operation of moving the operation handle 51 up and down, the same operation as an operation of swinging the operation handle 51 shown in FIG. 11 or the like can be realized. In the configuration shown in FIG. 29, the position or shape of the operation handle 51 or the plane portion 50b is not limited to the configuration shown in FIG. 29.

### Explanation of References

1: endoscope system

2: endoscope

3: light source device

4: processor

5: monitor

6: endoscope insertion part

7: endoscope operation part

8: universal cord

9: connector

10: endoscope distal end part

11: endoscope bendable part

12: endoscope connecting part

13: first treatment tool insertion port

14: first treatment tool channel

15: second treatment tool insertion port

16: second treatment tool channel

20: treatment tool for endoscope

21: insertion part

22: operation part

23: distal end part

24: grip part

25: bendable part

26: connecting part

27: operation wire

30, 30a, 30b: grip claw

31, 57, 81a, 81b: link member

32: support

33, 46, 82b: pin

34: guide groove

42: bending piece

43: outer coat

47: wire guide

50: operation part main body

50b: plane portion

51: operation handle

51a: free end part

52: fixing part

52a: connection metal fitting

55a, 55b, 55c: rotational movement support shaft part

56: wire holding part

60: forceps

61: claw

70: friction adjustment mechanism

83: connecting member

84: rotation shaft portion

85: insertion hole

86: extending portion

What is claimed is:

1. A treatment tool for an endoscope, comprising:
an insertion part that is insertable into a body and that includes a distal end part having an openable and closable treatment part;
an operation part that is disposed on a base end side of the insertion part; and
at least one operation wire that is connected to the operation part and that operates opening and closing of the treatment part,
wherein the treatment part includes a distal end operation part, a link member that is rotationally connected to a base end side of the distal end operation part, and a connecting member that is rotationally connected to a base end side of the link member,
the operation wire is fixed to the connecting member, and
at least a part of the operation wire and the link member overlap each other in a longitudinal axis direction of the insertion part.

2. The treatment tool for an endoscope according to claim 1,
wherein the connecting member has a rotation shaft portion that rotationally connects the link member and the connecting member.

3. The treatment tool for an endoscope according to claim 2,
wherein the connecting member has the rotation shaft portion and an insertion hole through which the operation wire is insertable and which intersects with a rotation axis of the rotation shaft portion, and
the operation wire is fixed to an inner surface of the insertion hole.

4. The treatment tool for an endoscope according to claim 3,
wherein the insertion hole is longer than the rotation shaft portion in the longitudinal axis direction.

5. The treatment tool for an endoscope according to claim 4,
wherein a portion where the inner surface of the insertion hole and the operation wire are in contact with each other is longer than the rotation shaft portion in the longitudinal axis direction.

6. The treatment tool for an endoscope according to claim 2, further comprising:
a housing that houses the connecting member and that has a guide groove for guiding a movement of the rotation shaft portion in the longitudinal axis direction.

7. The treatment tool for an endoscope according to claim 1,
wherein the insertion part includes a bendable part that is bendable and that is provided to be adjacent to the distal end part on a side of the operation part.

8. The treatment tool for an endoscope according to claim 7,
wherein the bendable part is bent by an operation of the operation part.

9. The treatment tool for an endoscope according to claim 1,
wherein the connecting member and the operation wire are fixed by laser welding.

10. The treatment tool for an endoscope according to claim 9,
wherein the connecting member and the operation wire are laser-welded at a location other than on a rear end surface of the connecting member.

11. An endoscope system comprising:
the treatment tool for an endoscope according to claim 1; and
an endoscope having a treatment tool channel through which the insertion part is insertable.
